(19) United States
(12) Patent Application Publication
(10) Pub. No.: US 2008/0214984 A1
(43) Pub. Date: Sep. 4, 2008

(54) PESSARY AND LUBRICANT SYSTEM AND METHOD

(76) Inventors: David Joseph Caracci, Cincinnati, OH (US); Debra Lynn Hartsell, Cincinnati, OH (US); Frank Delmar Macaulay, Cincinnati, OH (US); Michael Wayne Mason, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 12/034,858

(22) Filed: Feb. 21, 2008

Related U.S. Application Data
(60) Provisional application No. 60/902,413, filed on Feb. 21, 2007.

(51) Int. Cl. A61F 6/12; A61F 13/28

(52) U.S. Cl. 604/12; 128/834; 401/132

(57) ABSTRACT

A device comprising a tubular applicator and lotion dispenser in combination. The device includes a tubular applicator and a lotion dispenser for applying lotion to the tubular applicator. The lotion dispenser includes a lotion selected from the group consisting of gels, solutions, compositions, lubricants, and creams.

### CROSS REFERENCE TO RELATED APPLICATION

This application claims the benefit of U.S. Provisional Application Ser. No. 60/902,413 filed Feb. 21, 2007.

### FIELD OF INVENTION

This application relates to pessary devices for relief of female incontinence. More particularly, the present invention relates to pessary devices having a lotion coating on a surface thereof, and to systems for application of a lotion coating to either or both of the pessary device or the body.

### BACKGROUND OF THE INVENTION

Urinary incontinence, in which the ordinary bodily muscle functions fail to prevent unintended leakage of urine, is a common malady among women, particularly older women. It is estimated that up to 50% of women occasionally leak urine involuntarily, and that approximately 25% of women will seek medical advice at some point in order to deal with the problem. Stress incontinence, the most common type of urinary incontinence, refers to the involuntary loss of urine resulting from abdominal pressure rise, occurring during exercise, coughing, sneezing, laughing, etc. When stress incontinence occurs, it is usually the result of the abnormal descent of the urethra and bladder neck below the level of the pelvic floor. Many women wear sanitary napkins or diapers in order to deal with incontinence, and some women resort to surgical procedures.

Pessary devices are known to help relieve involuntary urination in a female. Such devices can be elastic and designed for arrangement in the vagina for compressive action on and support of the neck of the bladder. In some instances, pessary devices can elastically expand or unfold such that a portion presses against the anterior wall of the vagina and another portion presses against the posterior wall of the vagina, thereby bearing against the urethra.

One drawback to the use of pessary devices is the lack of comfort during insertion or withdrawal. Typically delivered using applicators similar to those used for tampons, pessary devices introduce discomfort during use due to their relatively larger size.

Another drawback to the use of pessary devices is the lack of natural lubrication in the vagina during use. Because pessaries can be used during non-menstrual periods, and for post-menopausal uses, dryness and friction during insertion and removal can be a problem.

Even for tampon uses, first time users and use during times of light loading can result in insertion and removal discomfort.

It would be beneficial to have a treatment for pessary devices that can enable reduce discomfort during insertion.

Additionally, it would be beneficial to have a treatment method and device for reducing discomfort during withdrawal of a pessary device.

Further, it would be beneficial to have a method and system for easing discomfort due to insertion or withdrawal, with the method and system being easy and discreet to use, transport, and dispose.

### SUMMARY OF THE INVENTION

A device comprising a tubular applicator and lotion dispenser in combination is disclosed. The device includes a tubular applicator and a lotion dispenser for applying lotion to the tubular applicator. The lotion dispenser includes a lotion selected from the group consisting of gels, solutions, compositions, lubricants, and creams.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is an exploded perspective view of a first embodiment of an pessary applicator according to the present invention.

FIG. 2 is a perspective view of an embodiment of an pessary for use with the applicators of the present invention.

FIG. 3 is a perspective view of an embodiment of an pessary for use with the applicators of the present invention.

FIG. 4 is a front view of the an embodiment of a pessary applicator, housing the pessary of FIG. 2.

FIG. 5 is a perspective view of an embodiment of the pessary applicator in the pessary housing position, and also shows the pessary member of FIG. 2 in phantom.

FIG. 6 is a perspective view of an embodiment of the pessary applicator in the pessary discharging position, and also shows the pessary member of FIG. 2 discharged from the pessary applicator.

FIG. 7 is a front view of an embodiment of the pessary applicator, housing the pessary of FIG. 2.

FIG. 8 is a perspective view of a pessary applicator and a lotion dispenser, showing lotion on both the dispenser and on the insertion end of the applicator.

FIGS. 9-12 show various embodiments of lotion dispensers.

### DETAILED DESCRIPTION OF THE INVENTION

The present invention is directed to pessary devices, or pessaries, which are inserted into the female vagina and expandable such that pressure is exerted on the urethra to help control involuntary urinary incontinence. In general, pessaries are delivered into the vagina in a similar manner as are tampons, by use of a plastic or cardboard tubular applicator comprising an outer member and in inner member, the inner member sometimes described as a plunger or an actuator, and used to push the pessary out of an open end of the outer member. In general, the improvement of the invention can be enjoyed for tampons and tampon applicators. Therefore the description below of a pessary device is not to be limiting to the full scope of the invention, as indicated by the claims herein.

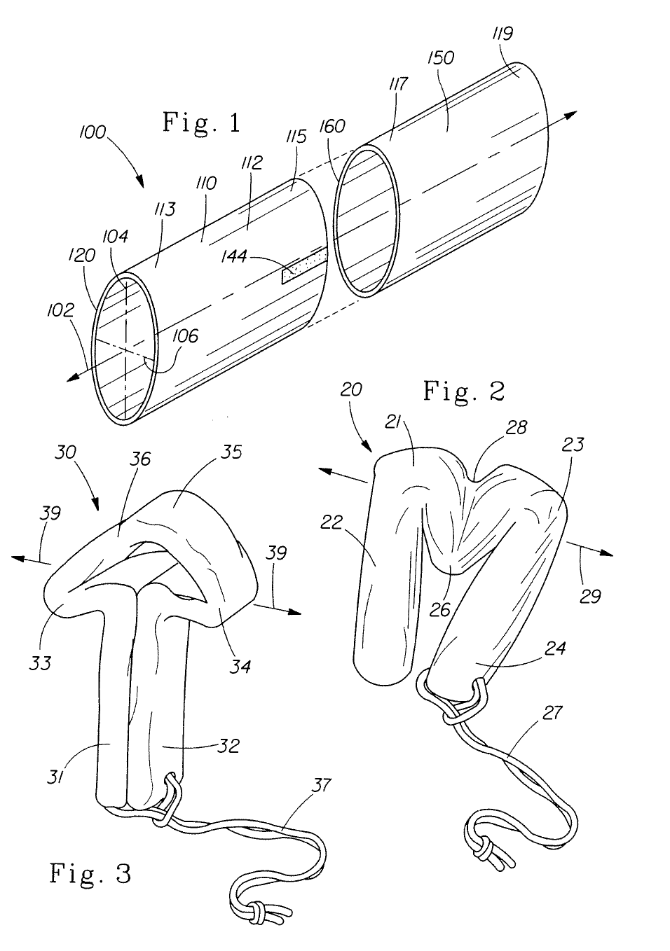

Referring now to the drawings and initially to FIG. 1, a pessary applicator is shown generally at 100 in a pre-assembled configuration. The applicator 100 has an outer member 110 defined by an pessary carriage or pessary housing, and an inner member 150 that can be referred to as a plunger or actuator. The outer member 110 can be in the shape of a hollow cylinder 112 extending the entire longitudinal length of the outer member 110, that is, from a leading, vaginal insertion end portion 113 to a trailing end portion 115. The cylinder 112 can extend substantially parallel to a longitudinal axis 102 of the applicator 100. In a direction substantially perpendicular to its longitudinal axis 102, the outer member 110 can be circular, or can be a non-circular, substantially elliptical or oval cross-section having a major axis 104 defined by the longest diameter of the cross-section, and a minor axis 106 defined by the shortest diameter of the cross-section. The outer member can have finger grips, flanges, grooves or indentions as is known in the art of tampon applicators. The outer member 110 houses a pessary (not shown) that is to be discharged into the vaginal cavity.

The inner member 150 can share the common longitudinal axis 102 with the outer member 110, and extends from an insertion end portion 117 to a trailing end portion 119. The inner member 150 is sized and adapted to be moveable within the outer member 110, and is operable to discharge the pessary from a pessary discharging exit end 120 of the outer member 110. The pessary discharging exit end 120 is the leading end of the applicator 100 when the applicator is inserted into the vagina. Preferably, as shown, the inner member 150 has a similar cross-section as the outer member, and can also have a substantially oval or elliptical cross-section in a direction substantially perpendicular to its longitudinal axis 102. The cross-section of the outer member 110 can be oval or elliptical (or substantially elliptical) rather than circular so as to more accurately approximate the shape of the vaginal opening, thereby facilitating the insertion of the applicator 100 into the vagina without discomfort to the user, while at the same time providing a volume that maximizes the space for housing a pessary member.

The applicator 100 can also include one or more orientation indicators. In the embodiment shown in FIG. 1, the orientation indicator is in the form of a marker 144, and is located on the outer surface of the outer member 110. The marker 144 is positioned so as to indicate the relative rotational position of the applicator 100, and hence, allows the user to correctly orient the pessary inside the vagina. In the embodiment shown, the marker 144 is located on the minor axis of the elliptical cross-section of the outer member 110.

FIGS. 2 and 3 illustrate exemplary pessary devices. A pessary device, or more particularly an “incontinence pessary device” as used herein refers to devices specifically designed, configured, and/or adapted for placement into a vagina in order to reduce the occurrence and/or severity of female urinary incontinence. While pessary devices are typically made of non-absorbent materials, at least partially absorbent materials or absorbent materials may also be used. Examples of pessary devices that can be employed in the present invention are shown in WO 2005/087154 published 22 Sep. 2005; WO 2004/103213, published 2 Dec. 2004; U.S. Pat. No. 6,090,038 granted 18 Jul. 2000; U.S. Pat. No. 5,618,256 granted 8 Apr. 1997; and U.S. Pat. No. 6,739,340 granted 25 May 2004.

In FIG. 2, a pessary member having a “M-shape” profile in a plane parallel to the directions indicated by arrows 29, is shown generally at 20. Pessary 20 can be formed from an elongated member having a first end portion 22 and a second end portion 24, and has three folds, 21, 23, and 26. The first end portion 22 and the second end portion 24 are aligned generally adjacent each other and, together with the folds 21, 23, 26, form a generally planar M-shaped profile. An inward crease 28 is formed in a direction generally perpendicular to the plane of the M-shaped profile. One of the end portions is attached to a withdrawal member, such as a string 27. The pessary member 20 can include, or can be made from, a resilient material that functions to expand the pessary 20 in at least a direction indicated by arrows 29 once the pessary 20 is placed within the vagina.

In order for the pessary 20 to be most effective in reducing the occurrence and/or severity of incontinence, the pessary 20 should be properly placed and correctly oriented within the vagina. Specifically, the pessary 20 should expand in the direction 29 so as to press against and/or compress the urethra and support the urinary sphincter muscle, thereby reducing the occurrence and/or severity of incontinence.

FIG. 3 illustrates another embodiment of a pessary useful for reducing the occurrence and/or severity of incontinence. Briefly, the pessary 30 can be formed from an elongated member that includes a first end portion 31 and a second end portion 32, and a connecting portion 36. The first end portion 31 and the second end portion 32 are aligned adjacent each other, and the connecting portion 36 includes three folds 33, 34, 35 in order to form a generally planar closed loop dome-shaped profile. The dome-shaped profile is in a plane parallel to the direction indicated by arrows 39. In this embodiment, both end portions can be attached to a withdrawal member, such as a string 37. The pessary 30 can include, or is can be made from, a resilient material that functions to expand the pessary 30 in at least a direction indicated by arrows 39 once the pessary 30 is placed within the vagina. Like the pessary of FIG. 2, the pessary 30 should be properly placed and oriented within the vagina to maximize its effectiveness. The pessary 30 can expand in the direction 39 so as to press against and/or compress the urethra and support the urinary sphincter muscle, thereby reducing the occurrence and/or severity of incontinence.

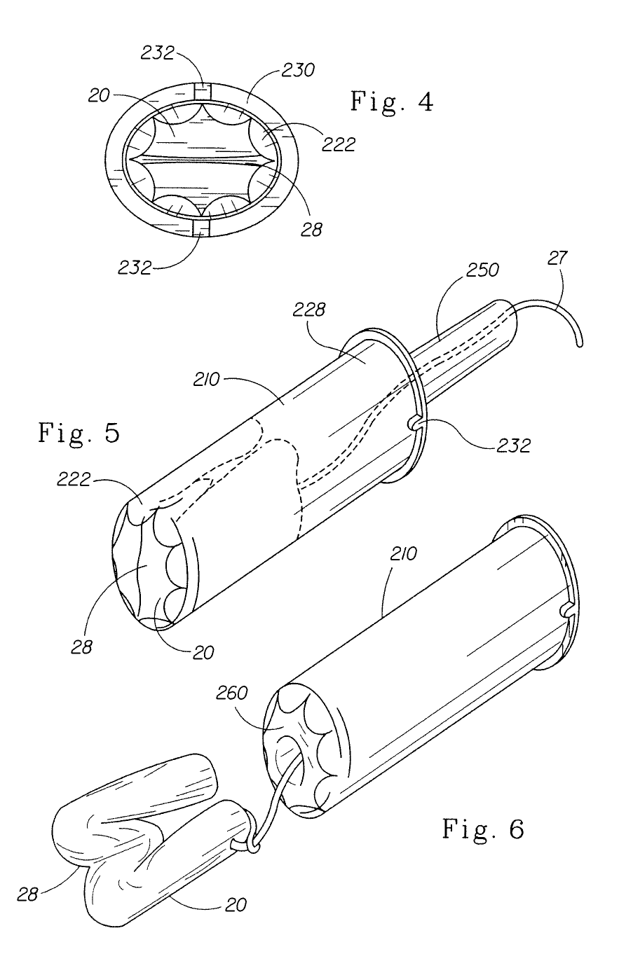

FIGS. 4 to 6 illustrate an applicator 200 with the pessary 20 of FIG. 2. FIG. 4 illustrates a front cross-sectional view of the applicator 200, shown housing the pessary 20. FIG. 5 illustrates the pessary 20 in phantom within the outer member 210, and inner member 250. FIG. 6 illustrates the pessary discharged from the outer member 210 after inner member 250 has been pushed into outer member 210. As shown, the inward crease 28 of the pessary 20 is substantially parallel to the major axis of the elliptical cross-section, and substantially perpendicular to the minor axis of the elliptical cross-section and an imaginary line connecting the orientation indicators 232. The pessary 20 is therefore oriented within the outer member 210 such that the plane defined by the M-shaped profile of the pessary 20 is substantially parallel to the minor axis of the substantially elliptical cross-section.

Those skilled in the art will recognize that applicator shapes and sizes and pessary shapes and sizes can be varied as desired to meet the incontinence needs of the user. Those skilled in the art will appreciate that various modifications may be made to the pessary devices shown. For example, the applicator may be round in cross section, and the pessary may be an un-folded, expandable device. Likewise, the manners of attachment of the withdrawing member shown in FIGS. 2 and 3 are only exemplary, and other manners known in the art can be used equally as well. Likewise, those skilled in the art will appreciate that various modifications may be made to applicators 100 and 200, such as modifications known in the art of tampon insertion.

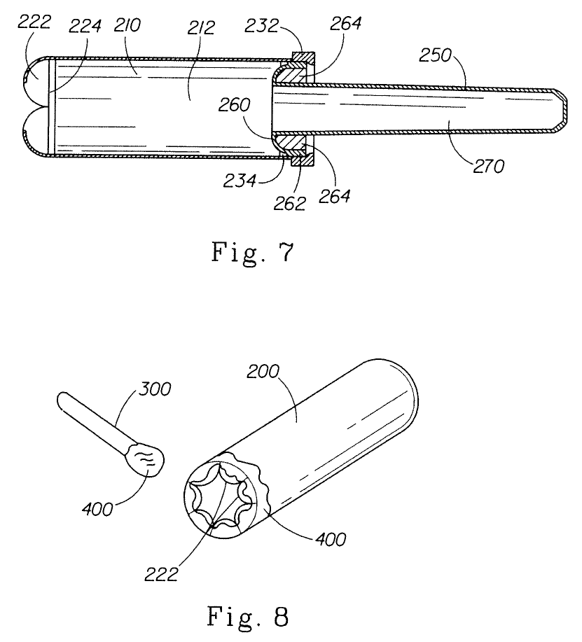

FIG. 7 illustrates the applicator 200 in a partial cutaway view of an assembled configuration. The inner member 250 of the applicator 200 includes a head portion 260, a shank portion 270 and a trailing portion 275. The head portion 260 rests in the trailing end 240 of the outer member 210 with the extensions 262 slideably engaging the inner surface of the outer member 210. The head portion 260 can be held in place longitudinally by a retaining rib 234. The applicator 200 can be formed of a material, such as a thermoplastic, that allows for slight deformation of the extensions 262 when the inner member 250 is pushed by the user, thereby allowing the extensions 262 to pass the retaining rib 234, and to allow the inner member 250 to advance through the interior of the outer member 210.

The head portion 260 can be integrally formed with or joined to the shank portion 270 by a continuous return flange 265 extending from the exit 290 back towards the trailing portion 275. The flange 265 is preferably reinforced by support members 264 connected with the outer surface of the shank portion 270. The flange 265 includes a plurality of outer member engaging portions defined by extensions 262 for contacting the interior of the outer member 210. Although the flange 265 shown extends a relatively short length relative to the full length of the inner member 250, in other embodiments, the flange 265 extends significantly longer relative to the length of the inner member 250.

The shank portion 270 can have a substantially smaller cross-section than the head portion 260. Because of this smaller cross-section, the user is able to actuate the inner member 250 with a single finger of the user's same hand that is holding the outer member 210 at the gripping portion 228. Furthermore, the smaller cross-section shank portion 270 is more aesthetically pleasing because it makes the applicator as a whole appear smaller and less likely to cause discomfort in its use.

The above description of a pessary and pessary applicator is provided as exemplary of devices benefiting by the improvement of the present invention. For a more full description of the exemplary pessary applicator, see U.S. Pat. No. 6,645,136, issued Nov. 11, 2003 to Zunker et al.

In one embodiment pessary 20 can be inserted by a non-tubular applicator. Pessary 20 can be inserted by pushing into the vagina with a rigid insertion member, such as a wooden stick, plastic stick, or other suitable member. Such pessaries and insertion devices are disclosed in U.S. Pat. No. 5,618,256, granted 8 Apr. 1997. Therefore, in one embodiment, an applicator can be a device that does not surround the pessary prior to use, but is an applicator that acts to push the pessary in, much like the inner member of the above-described pessary applicators 100 or 200.

The present invention is an improvement in the art of pessary devices by providing in combination with a pessary insertion device such as applicator 200, a lotion application device, and a method and system for application of a lotion to either the applicator 100 or 200, or the body of the user. As shown in FIG. 8, lotion 400 can be dispensed from a lotion dispenser 300 onto the insertion end tip of applicator 200, including the petals 222. Lotion 400 on the tip of an applicator 200 can aid in minimizing or eliminating the discomfort associated with insertion of the applicator into the vagina for proper insertion of the pessary into the vagina. Although useful for tampon applicators, the benefit can be enjoyed more for pessary applicators because pessaries are intended to be placed higher in the vagina, such that the applicator may need to be inserted farther into the vagina than a woman is comfortable with in the un-lotioned condition.

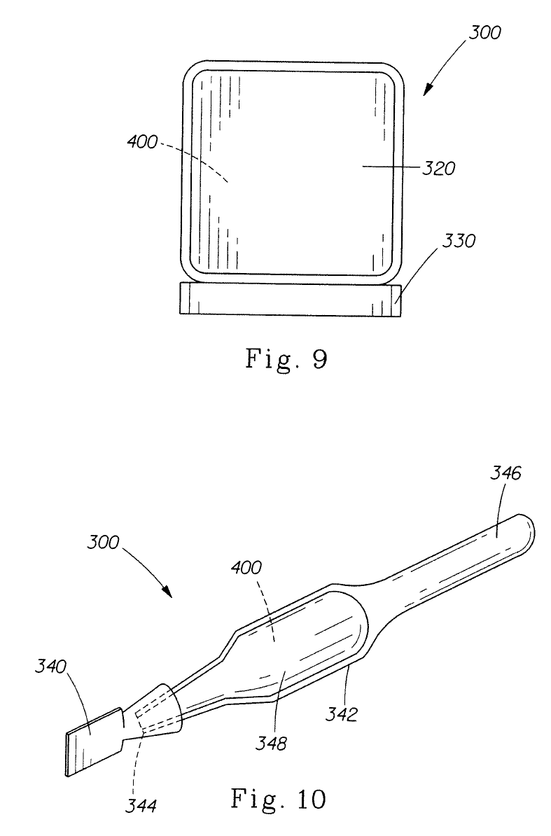

As shown in FIG. 9, a lotion dispenser 300 in the form of an enclosed pouch can be utilized to dispense lotion on the tip of applicator 200, such as onto the flanges defining petals 222, which can soften the vaginal insertion process. Lotion dispenser 300 can dispense a lotion 400, which can be any of gels, solutions, compositions, lubricants, creams, or other lotions known to ease the discomfort caused by the introduction of an object into the vagina. Exemplary lotions include those intended to reduce frictional discomfort, including those of the type marketed by or similar to K-Y® brand jelly and lubricants. Other lubricants include solid microspheres, mineral oil, and Surgilube® chlorhexidine gluconate.

Lotion dispenser 300 can be any of known devices utilized to dispense viscous fluids. For example, lotion dispenser 300 can be of the type currently marketed by The Procter and Gamble Co. as Olay® brand eye derma-pod. Such as device is shown in FIG. 9, and can include a flexible foil pouch 320, sealed about the edges and containing therein a suitable lotion or cream for vaginal use. Upon squeezing a sufficient between the finger and thumb with a sufficient force, the pouch 320 breaks and lotion or cream is released into a soft, resilient spongy tip 330, which can be a foam sponge applicator. In use, the user can squeeze lotion, cream, gel, or other lubricant into soft, resilient spongy tip 330 as necessary to rub or wipe onto applicator 200 or onto the body portion to be contacted by applicator 200. Pouch 320 can serve as a handle to provide a finger hold portion such that the user can apply lotion to either the tip of an applicator, to her body, or both prior to, or after insertion of pessary device. The size, length, and overall dimensions of applicator 300 shown in FIG. 9 can be tailored for use as necessary. In one embodiment, for example, the dimension of soft, resilient spongy tip 330 can be sized such that it extends beyond the largest dimension of pouch 320 so that discomfort due to the pouch pushing on any body parts is minimized or eliminated. In one embodiment, the overall size is approximately 2.5 cm on a side and can be folded to be generally flattened for storage and shipping. The pouch 320 and spongy tip 330 can be adhered together by means known in the art, and the entire dispenser can be separately packaged in a separate wrapper or box.

Lotion dispenser 300 can be of the type currently marketed by Alcon® as Tears Naturale Free®, which is a generally rigid, plastic device depicted in FIG. 10. As shown, dispenser 300 can have a removable and reusable cap 340 that can be frangibly joined to body 342 which can be integral with handle 346. Between the capped tip and the handle is a reservoir portion 348 from which lotion, cream, gel, or other lubricant 400 can be squeezed out. In use, the user can twist off cap 340 and squeeze lotion or lubricant 400 from open tip 344. Handle 346 provides a finger hold portion such that the user can use the dispenser to apply lotion to either the tip of an applicator, to her body, or both prior to or after insertion of pessary device. The size, length, and overall dimensions of applicator 300 shown in FIG. 10 can be tailored for use as necessary. In one embodiment, the overall length is about 8-10 cm and the handle portion is about 2-3 cm.

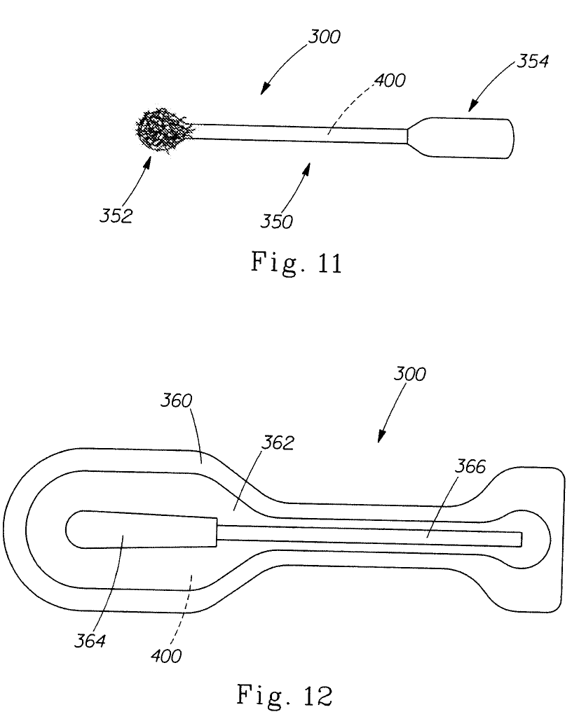

Lotion dispenser 300 can be of the type currently marketed as Baby Orajel® teething swabs, as depicted in FIG. 11. As shown, dispenser 300 can have a hollow round tube 350 that is frangibly connected and openable to a cotton-swabbed tip 352. In use, the user can squeeze the other end 354 of dispenser 300 to cause pressure to break a frangible seal between lotion, cream, gel, or other lubricant 400 inside tube 350 and cotton-swabbed tip 352, thereby permitting lotion to be absorbed into or forced into cotton-swabbed tip 352. Hollow tube 350 provides a finger hold portion such that the user can apply lotion to either the tip of an applicator, to her body, or both prior to, or after insertion of pessary device. The size, length, and overall dimensions of applicator 300 shown in FIG. 11 can be tailored for use as necessary. In one embodiment, the overall length of tube 350 can be about 8-10 cm, and the diameter of a tube having a round cross section can be from about 1 to about 5 mm. Tube materials can include any plastic materials as known in the art. In one embodiment, tube 350 can be transparent, such that lotion 400 quantity can be observed.

Lotion dispenser 300 can be of the type currently marketed as Zicam® No-Drip Liquid Nasal Gel, as depicted in plan view in FIG. 12. As shown, dispenser 300 can have a foil backing layer 360 to which a clear plastic bubble packaged element 362 is bonded and which encloses a Q-tip®-like swab 364. A lotion, cream, gel, or other lubricant 400 is also enclosed with swab 364. In use, the user can open the dispenser 300 by tearing the plastic bubble element away from the foil backing and removing swab 364. The “stick” portion 366 of swab 364 provides a finger hold portion such that the user can apply lotion to either the tip of an applicator, to her body, or both prior to, or after insertion of pessary device. The size, length, and overall dimensions of applicator 300 shown in FIG. 12 can be tailored for use as necessary. In one embodiment, the foil backing 360 can have a length of about 4-8 cm, and a width of about 3-3 cm. The swab 364 can have dimensions typical of swabs, including Q-tip® swabs.

Lotion dispenser 300 can be of the type currently marketed as 3M Cavilon® No Sting Barrier Film, which is a swab type plastic dispenser.

In one embodiment, lotion dispenser 300 can include commonly available items such as a simple swab, such as a Q-tip® swab marketed by Johnson Diversey. Lotion dispenser can also incorporate cotton balls, sponges, wipes, or items useful for spreading fluid or viscous fluids onto a surface.

The method of the present invention includes providing an applicator, such as a tampon applicator or pessary applicator. Applicator can be a plastic or cardboard tubular applicator, such as applicators 100 and 200 shown herein. The method also includes providing a lotion, cream, or other suitable lubricant 400 that can be utilized to lessen the discomfort associated with insertion or removal of a pessary, including the frictional discomfort caused by the applicator during insertion, and the general discomfort caused by removal of an expanded pessary after use. For insertion, the user can dispense by squeezing, wiping, or otherwise placing lotion on or near the tip or insertion end of the applicator, and can also put lotion on any other portion of the applicator or vaginal area thought necessary for increasing comfort of insertion. For withdrawal, the user can squeeze, wipe, or otherwise place lotion in, on or near the vaginal opening, and can also put lotion on any other portion of the body thought necessary for increasing comfort of withdrawal of the pessary device.

Lotion dispensers 300 can be packaged with pessary devices 20 or packaged separately. If packaged with pessary devices, dispensers 300 can be joined to the pessary device, such as by being adhered to the packaging of the device, or dispensers 300 can be enclosed in the packaging of the pessary device. In another embodiment, dispensers can be separately packaged, such as in a separate openable and sealable container. In one embodiment pessary devices can be individually packaged, and an individually packaged lotion dispenser can be associated each individually packaged pessary device. In one embodiment, individually packaged lotion dispensers can be packaged loosely with individually wrapped pessary devices. In one embodiment individually wrapped lotion dispensers can be adhered by adhesive, tape, or other joining means, to the packaging of individually wrapped pessary devices.

Lotion dispensers can be packaged in reusable, openable carrying cases, such as soft or rigid plastic cases that can be carried in a purse or handbag.

The above disclosure applies to tampons and tampon applicators as well. For either tampon applicators or pessary applicators, starter kits for new users can include combinations of applicators and lotion dispensers. In some embodiments, two lotion dispensers can be packaged with each applicator, whereby a first lotion can first be dispensed onto applicator, and later a second lotion, which may be different than the first lotion, can be dispensed onto the vaginal opening for removal of the tampon or pessary device.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A device comprising a tubular applicator and lotion dispenser in combination, the device comprising:
a. an applicator; and
b. a lotion dispenser for applying lotion to said tubular applicator, said lotion dispenser comprising a lotion selected from the group consisting of gels, solutions, compositions, lubricants, and creams.

2. The device of claim 1, wherein said applicator is a pessary device and encloses a pessary.

3. The device of claim 1, wherein said applicator is generally tubular in shape.

4. The device of claim 1, wherein said applicator is generally rigid and in the shape of a stick.

5. The device of claim 1, wherein said lotion dispenser is a first lotion dispenser, and the device further comprises a second lotion dispenser.

6. The device of claim 5, wherein said first lotion dispenser comprises a first lotion, and said second lotion dispenser comprises a second lotion that is different from said first lotion.

7. The device of claim 1, wherein said applicator is a tampon device and encloses a tampon.

8. The device of claim 1, wherein said lotion dispenser further comprises frangible openings made to be opened by the application of finger pressure.

9. The device of claim 1, wherein said lotion dispenser further comprises a swab.

10. The device of claim 1, wherein said lotion dispenser further comprises a hollow tube comprising said lotion, said hollow tube having at one end a soft swab.

11. The device of claim 1, wherein said lotion dispenser further comprises a foil pouch.

12. A method for preparing a pessary device for inserting a pessary into the vagina, said method comprising:
a. providing an applicator having an insertion end;
b. providing a lotion dispenser comprising a lotion; and
c. dispensing said lotion on said insertion end of said applicator.

13. The method of claim 12, wherein said lotion dispenser further comprises frangible openings made to be opened by the application of finger pressure.

14. The method of claim 12, wherein said lotion dispenser further comprises a swab.

15. The method of claim 12, wherein said lotion dispenser further comprises a hollow tube comprising said lotion, said hollow tube having at one end a soft swab.

16. The method of claim 12, wherein said lotion dispenser further comprises a foil pouch.
